(19) United States
(12) Patent Application Publication
(10) Pub. No.: US 2006/0105440 A1
(43) Pub. Date: May 18, 2006

(54) Method of producing polyhydroxyalkanoate

(76) Inventors: Koichi Kinoshita, Kakogawa-shi (JP); Yoshifumi Yanagida, Akashi-shi (JP); Fumio Osakada, Okayama-shi (JP); Yasuyoshi Ueda, Himeji-shi (JP)

Correspondence Address: KENYON & KENYON LLP, 1500 K STREET N.W., SUITE 700, WASHINGTON, DC, 20005, US

(21) Appl. No.: 11/237,686

(22) Filed: Sep. 29, 2005

Related U.S. Application Data
(60) Provisional application No. 60/628,116, filed on Nov. 17, 2004.

(30) Foreign Application Priority Data
Sep. 30, 2004 (JP) 2004-287457

(51) Int. Cl. C12P 7/62

(52) U.S. Cl. 435/135

(57) ABSTRACT

The present invention has its object to provide a method of producing PHA by extracting, separating, and purifying PHA from biomass containing PHA having an weight average molecular weight of more than 2,000,000, by which smooth stirring can be carried out during extraction and filterability of a residue becomes good to thereby efficiently produce PHA with good operability. In the present invention, PHA is extracted, separated, and purified from biomass by a method of producing polyhydroxyalkanoate by extracting and isolating polyhydroxyalkanoate by using an aprotic organic solvent from biomass containing polyhydroxyalkanoate having a weight average molecular weight of more than 2,000,000, which comprises decreasing the weight average molecular weight of the polyhydroxyalkanoate through any one of the following treatments: (a) the biomass is heated at 40 to 500° C. before addition of the aprotic organic solvent; (b) the biomass is heated at 40 to 500° C. before addition of the aprotic organic solvent, and is further heated at 40 to 200° C. in the aprotic organic solvent; (c) the biomass is heated at 40 to 500° C. before addition of the aprotic organic solvent, and is further heated at 40 to 200° C. in the aprotic organic solvent in the presence of water and/or an alcohol; (d) the biomass is not heated before addition of the aprotic organic solvent, but is heated at 40 to 200° C. in the aprotic organic solvent; and (e) the biomass is not heated before addition of the aprotic organic solvent, but is heated at 40 to 200° C. in the aprotic organic solvent in the presence of water and/or an alcohol.

### CROSS-REFERENCE TO RELATED APPLICATIONS

This application claims priority to JP 2004-287457 filed 30 Sep. 2004 and to U.S. Provisional Application No. 60/628,116 filed 17 Nov. 2004.

### TECHNICAL FIELD

The present invention relates to a method of producing polyhydroxyalkanoate by extracting and isolating polyhydroxyalkanoate by using an aprotic organic solvent from biomass containing polyhydroxyalkanoate, wherein the weight average molecular weight of polyhydroxyalkanoate is decreased by heat treatment or by the use of an additive to efficiently produce polyhydroxyalkanoate with good operability.

### BACKGROUND ART

Polyhydroxyalkanoate (hereinafter abbreviated as “PHA”) is a biodegradable thermoplastic polyester that many species of microorganisms synthesize and accumulate within cells as the energy storage materials. PHA produced by microorganisms using natural organic acids or fats and oils as carbon sources is completely biodegraded by microorganisms in soil or water, and is therefore incorporated in a natural carbon cycle process. For this reason, PHA can be said to be an environment-conscious plastic material which hardly affects on ecosystems adversely. In recent years, synthetic plastics have caused serious social problems in the viewpoints of environmental pollution, waste disposal, and oil resources. Therefore, PHA attracted attention as an ecofriendly plastic good for the environment, and it is strongly desired that PHA be put to practical use.

PHA can be industrially produced by microorganisms which can naturally produce PHA or by transformants obtained by introducing a PHA synthetic enzyme gene into microorganisms or plants as hosts. In both cases, PHA is accumulated in such biomass, and therefore PHA can be obtained by recovering the biomass containing PHA and then separating and purifying PHA from the biomass.

PHA can be separated and purified from biomass by extracting PHA from biomass with a solvent which can dissolve PHA, adding a poor solvent thereto to crystallize PHA, and then recovering crystalline PHA, which is known as the simplest method of separating and purifying PHA. For example, there is known a method in which biomass accumulating PHA is dried, PHA is extracted from the dried biomass with a halogen-based organic solvent such as chloroform or methylene chloride, an extraction residue is separated by filtration, and then a resultant extraction liquid is mixed with a poor solvent such as methanol or hexane to precipitate and recover PHA (see Japanese Kokai Publication Sho-59-205992). U.S. Pat. No. 5,942,597 describes a method in which PHA is extracted with a solvent such as acetone, acetonitrile, or toluene. Japanese Kohyo Publication Hei-10-504460 describes a method wherein PHA is extracted with a solvent such as lower ketone or dialkyl ether. The present inventors have carried out supplementary tests on these methods, and as a result they have found that only when biomass containing PHA having an average molecular weight of more than 2,000,000 is used, it is impossible to stir a liquid during extraction because of its extremely high viscosity and the filterability is very poor so that it is substantially impossible to separate an extraction residue by filtration. In Examples of Japanese Kokai Publication Hei-2-69187, the average molecular weight of PHA is slightly decreased when PHA is extracted with a solvent such as diester of succinic acid or butyrolactone. However, in these Examples, PHA having an average molecular weight of 1,000,000 or less is used, and therefore in this document there is no description, as its aim or object, that it is impossible to stir a liquid during extraction because of its extremely high viscosity or the filterability is very poor when an extraction residue is separated by filtration. Further, Japanese Kohyo Publication Hei-10-504460 describes that PHA is extracted with an alcohol such as isopropanol or hexanol, and Japanese Kokai Publication Hei-11-511025 describes that PHA is extracted with a solvent such as acetone, acetic ester, or toluene. In both of the patent documents, there is no description about the average molecular weight of PHA, but it can be considered that PHA having an average molecular weight of 2,000,000 or less is used because there is no description, as its aim or object, that it is impossible to stir a liquid during extraction because of its extremely high viscosity or the filterability is very poor when an extraction residue is separated by filtration.

As described above, it has not been known at present that it is substantially impossible to carry out extraction and purification of PHA from biomass containing PHA having an average molecular weight of more than 2,000,000.

### SUMMARY OF THE INVENTION

Therefore, an object of the present invention is to provide a method of producing PHA by extracting, separating, and purifying PHA from biomass containing PHA having an average molecular weight of more than 2,000,000, by which smooth stirring can be carried out during extraction and filterability of an extraction residue becomes good to thereby efficiently produce PHA with good operability.

### DETAILED DESCRIPTION OF THE INVENTION

The present inventors have intensively investigated to achieve the above object, and as a result they have found that it is possible to industrially produce PHA with good productivity by heating biomass containing PHA having a weight of average molecular weight of more than 2,000,000, and/or by heating biomass containing PHA having a weight average molecular weight of more than 2,000,000 when PHA is extracted from the biomass with an aprotic organic solvent, and by further heating the same in the presence of water and/or an alcohol in order to control the weight average molecular weight of PHA, that is, in order to decrease the weight average molecular weight of PHA to a desired level so that stirring can be carried out during extraction and filterability of an extraction residue becomes good. This finding has lead to the completion of the present invention.

The first invention relates to

a method of producing polyhydroxyalkanoate by extracting and isolating polyhydroxyalkanoate by using an aprotic organic solvent from biomass containing polyhydroxyalkanoate having a weight average molecular weight of more than 2,000,000,

which comprises decreasing the weight average molecular weight of the polyhydroxyalkanoate through any one of the following treatments (a) to (e):

(a) the biomass is heated at 40 to 500° C. before addition of the aprotic organic solvent;

(b) the biomass is heated at 40 to 500° C. before addition of the aprotic organic solvent, and is further heated at 40 to 200° C. in the aprotic organic solvent;

(c) the biomass is heated at 40 to 500° C. before addition of the aprotic organic solvent, and is further heated at 40 to 200° C. in the aprotic organic solvent in the presence of water and/or an alcohol;

(d) the biomass is not heated before addition of the aprotic organic solvent, but is heated at 40 to 200° C. in the aprotic organic solvent; and

(e) the biomass is not heated before addition of the aprotic organic solvent, but is heated at 40 to 200° C. in the aprotic organic solvent in the presence of water and/or an alcohol.

Preferred embodiments of the first invention relate to

the method of producing polyhydroxyalkanoate defined above,

wherein the aprotic organic solvent is at least one species selected from the group consisting of aromatic hydrocarbons having 6 to 10 carbon atoms, ketones having 3 to 7 carbon atoms, and fatty acid alkyl esters having 4 to 8 carbon atoms;

the method of producing polyhydroxyalkanoate defined above,

wherein the aromatic hydrocarbons having 6 to 10 carbon atoms is at least one species selected from the group consisting of benzene, chlorobenzene, toluene, xylene, ethylbenzene, cumene, butylbenzene, cymene, and isomers thereof;

the method of producing polyhydroxyalkanoate defined above,

wherein the ketone having 3 to 7 carbon atoms is at least one species selected from the group consisting of acetone, methylethylketone, methylbutylketone, pentanone, hexanone, cyclohexanone, heptanone, and isomers thereof.

the method of producing polyhydroxyalkanoate defined above,

wherein the fatty acid alkyl ester having 4 to 8 carbon atoms is at least one species selected from the group consisting of ethylacetate, propylacetate, butylacetate, pentylacetate, hexylacetate, and isomers thereof;

the method of producing polyhydroxyalkanoate defined above,

wherein the biomass is heated for 1 minute to 240 hours before addition of the aprotic organic solvent;

the method of producing polyhydroxyalkanoate defined above,

wherein the biomass is heated in the aprotic organic solvent for 1 minute to 240 hours;

the method of producing polyhydroxyalkanoate defined above,

wherein the biomass is heated in the aprotic organic solvent in the presence of water and/or an alcohol for 1 minute to 240 hours;

the method of producing polyhydroxyalkanoate defined above,

wherein the amount of water and/or an alcohol is 0.01 to 70 parts by weight per 100 parts by weight of the aprotic organic solvent;

the method of producing polyhydroxyalkanoate defined above,

wherein the alcohol has 1 to 20 carbon atoms;

the method of producing polyhydroxyalkanoate defined above,

wherein the alcohol is at least one species selected from the group consisting of methanol, ethanol, propanol, butanol, pentanol, hexanol, heptanol, octanol, nonanol, decanol, and isomers thereof;

the method of producing polyhydroxyalkanoate defined above,

wherein the polyhydroxyalkanoate is a copolymer obtained by copolymerizing at least two monomers selected from the group consisting of 3-hydroxybutylate, 3-hydroxyvalerate, 3-hydroxypropionate, 4-hydroxybutylate, 4-hydroxyvalerate, 5-hydroxyvalerate, 3-hydroxyhexanoate, 3-hydroxyheptanoate, 3-hydroxyoctanoate, 3-hydroxynonanoate, and 3-hydroxydecanoate;

the method of producing polyhydroxyalkanoate defined above,

wherein the polyhydroxyalkanoate is a copolymer comprising 3-hydroxyhexanoate and at least one hydroxyalkanoate other than 3-hydroxyhexanoate;

the method of producing polyhydroxyalkanoate defined above,

wherein the polyhydroxyalkanoate is a copolymer comprising 3-hydroxyhexanoate and 3-hydroxybutylate;

the method of producing polyhydroxyalkanoate defined above,

wherein the polyhydroxyalkanaote is produced by a microorganism belonging to a genus selected from the group consisting of Alcaligenes, Azotobacter, Bacillus, Clostridium, Halobacterium, Norcadia, Rhodospirillum, Pseudomonas, Ralstonia, Zoogloea, Candida, Yarrowia, Saccharomyces, and Aeromonas;

the method of producing polyhydroxyalkanoate defined above,

wherein the polyhydroxyalkanoate is produced by a transformant into which a polyhydroxyalkanoate synthase gene cluster derived from Aeromonas caviae has been introduced; and

the method of producing polyhydroxyalkanoate defined above,

wherein the polyhydroxyalkanoate is produced by Ralstonia eutropha into which a polyhydroxyalkanoate synthase gene cluster derived from Aeromonas caviae has been introduced.

The second invention relates to

a method of producing an extraction residue by extracting and isolating polyhydroxyalkanoate by using an aprotic organic solvent from biomass containing polyhydroxyalkanoate having a weight average molecular weight of more than 2,000,000,

which comprises extracting the polyhydroxyalkanaote from the biomass through any one of the following treatments (a) to (e) to obtain an extraction residue:

(a) the biomass is heated at 40 to 500° C. before addition of the aprotic organic solvent;

(b) the biomass is heated at 40 to 500° C. before addition of the aprotic organic solvent, and is further heated at 40 to 200° C. in the aprotic organic solvent;

(c) the biomass is heated at 40 to 500° C. before addition of the aprotic organic solvent, and is further heated at 40 to 200° C. in the aprotic organic solvent in the presence of water and/or an alcohol;

(d) the biomass is not heated before addition of the aprotic organic solvent, but is heated at 40 to 200° C. in the aprotic organic solvent; and

(e) the biomass is not heated before addition of the aprotic organic solvent, but is heated at 40 to 200° C. in the aprotic organic solvent in the presence of water and/or an alcohol: and

decreasing the amount of the solvent contained in the extraction residue.

The third invention relates to

feed for animals, feed for microorganisms, and fertilizer for plants,

which contain an extraction residue produced as mentioned above.

Hereinafter, the present invention will be described in more detail. First, the first invention, that is, a method of extracting and isolating polyhydroxyalkanoate by using an aprotic organic solvent from biomass containing polyhydroxyalkanoate will be described.

<Method of Extracting and Isolating Polyhydroxyalkanoate>

Biomass to be used in the present invention is not particularly limited as long as it is an organism which can accumulate polyhydroxyalkanoate (PHA) within cells. For example, microorganisms belonging to the genus Alcaligenes such as Alcaligenes lipolytica and Alcaligenes latus, microorganisms belonging to the genus Ralstonia such as Ralstonia eutropha, and microorganisms belonging to the genera Pseudomonas, Bacillus, Azotobacter, Nocardia, Clostridium, Halobacterium, Rhodospirillum, Zoogloea, Candida, Yarrowia, Saccharomyces, and Aeromonas can accumulate PHA within cells under controlled culture conditions. Alternatively, transformants obtained by introducing a PHA synthesis-related gene cluster derived from the above-mentioned microorganisms may be used as microorganisms. In this case, examples of a host include, but are not limited to, the above-mentioned microorganisms, microorganisms such as Escherichia coli and yeast (see International Publication WO01/88144), plants and the like. Among them, Aeromonas caviae (hereinafter, abbreviated as “A. caviae”) belonging to the genus Aeromonas and transformants obtained by introducing the gene of PHA synthetic enzymes derived from A. caviae are preferably used because they have the ability to synthesize PHA which is excellent as a polymer. Particularly, Ralstonia eutropha into which the gene of PHA synthetic enzymes derived from A. caviae has been introduced is more preferably used. As one example of such microorganisms, Alcaligenes eutrophus AC32 has been internationally deposited under the Budapest Treaty with the International Patent Organism Depositary of the National Institute of Advanced Industrial Science and Technology located at Central 6, 1-1-1 Higashi, Tsukuba, Ibaraki, Japan (Date of original deposit: Aug. 12, 1996; Date of transfer: Aug. 7, 1997; Accession No. FERM BP-6038).

A method of cultivating the above-mentioned microorganism which can produce PHA is not particularly limited, but a method well known to those skilled in the art which is disclosed in, for example, Japanese Kokai Publication 2001-340078 can be employed. After the completion of cultivation, biomass is obtained in the usual manner. For example, a culture solution may be directly dried by, for example, spray drying to obtain dried biomass or a culture solution may be subjected to centrifugation or filter separation to recover biomass. Recovered biomass may be either dry or wet with water when subjected to an extraction process. Alternatively, wet biomass obtained by washing recovered microbial cells with a lipid solvent such as methanol or acetone or dried biomass obtained by drying such wet biomass may be used as biomass for PHA extraction.

Of course, the cultured microorganism preferably has a high PHA content. In industrial application, dried biomass preferably contains 50% by weight or more of PHA. In view of subsequent separating operation and the purity of a separated polymer, PHA content of dried biomass is more preferably 60% by weight or more, even more preferably 70% by weight or more.

The method of producing PHA according to the present invention makes it possible to easily extract and isolate PHA having a weight average molecular weight of more than 2,000,000 by decreasing the molecular weight of such PHA, and further is preferably applied to PHA having a molecular weight of more than 2,500,000. It is to be noted that in this specification, the weight average molecular weight is determined by gel chromatography using a gel chromatography system with RI detection (manufactured by Shimadzu Corporation) equipped with two columns of Shodex K806L (manufactured by Showa Denko Co., Ltd., 300×8 mm) connected in series and polystyrene as a molecular weight standard.

From biomass containing PHA, which is obtained in such a manner described above, PHA is extracted by adding an aprotic organic solvent to the biomass and then stirring them at a predetermined temperature for a predetermined time.

Examples of the aprotic organic solvent to be used in the present invention include aromatic hydrocarbons having 6 to 10 carbon atoms, ketones having 3 to 7 carbon atoms, fatty acid alkyl esters having 4 to 8 carbon atoms, halogen-based organic solvents such as chloroform and methylene chloride, and the like. Among these aprotic organic solvents, at least one species selected from the group consisting of aromatic hydrocarbons having 6 to 10 carbon atoms, ketones having 3 to 7 carbon atoms, and fatty acid alkyl esters having 4 to 8 carbon atoms is preferably used.

Examples of the aromatic hydrocarbon having 6 to 10 carbon atoms include benzene, chlorobenzene, toluene, xylene, ethyl benzene, cumene, butylbenzene, cymene, and isomers thereof (e.g., 1,2,3-trimethylbenzene, 1,2,4-trimethylbenzene, etc.).

Examples of the ketone having 3 to 7 carbon atoms include acetone, methylethylketone, methylbutylketone, pentanone, hexanone, cyclohexanone, heptanone, and isomers thereof (e.g., methyl-n-amylketone, methylisobutylketone, 2-hexanone, 3-hexanone, 5-methyl-2-hexanone, etc.).

Examples of the fatty acid alkyl ester having 4 to 8 carbon atoms include ethylacetate, propylacetate, butylacetate, pentylacetate, hexylacetate, and isomers thereof (e.g., isobutylacetate, isoamylacetate, isobutylisobutyrate, methylpropionate, ethylpropionate, propylpropionate, butylpropionate, pentylpropionate, methylbutyrate, ethylbutyrate, propylbutyrate, butylbutyrate, methylvalerate, ethylvalerate, etc.).

At least one of these aprotic organic solvents can be used. Among these aprotic organic solvents, aromatic hydrocarbons having 6 to 10 carbon atoms and ketones having 3 to 7 carbon atoms are more preferably used. Even more preferably, toluene, benzene, chlorobenzene, acetone, methylethylketone, butylacetate, and butylpropionate are used because PHA is highly soluble in these aprotic organic solvents. Among them, toluene is particularly preferred because it is relatively cheap.

In the method of producing PHA according to the present invention, any one of the following treatments (a) to (e) is carried out to control the weight average molecular weight of PHA so as to decrease the molecular weight of PHA to a desired level, when PHA is extracted and isolated from biomass containing PHA by adding an aprotic organic solvent to the biomass:

(a) the biomass containing PHA is heated at 40 to 500° C. before addition of an aprotic organic solvent;

(b) the biomass containing PHA is heated at 40 to 500° C. before addition of an aprotic organic solvent, and is further heated at 40 to 200° C. in the aprotic organic solvent;

(c) the biomass containing PHA is heated at 40 to 500° C. before addition of an aprotic organic solvent, and is further heated at 40 to 200° C. in the aprotic organic solvent in the presence of water and/or an alcohol;

(d) the biomass containing PHA is not heated before addition of an aprotic organic solvent, but is heated at 40 to 200° C. in the aprotic organic solvent; and

(e) the biomass containing PHA is not heated before addition of an aprotic organic solvent, but is heated at 40 to 200° C. in the aprotic organic solvent in the presence of water and/or an alcohol.

<Treatment (a)>

Biomass obtained in such a manner described above is heated before addition of an aprotic organic solvent to control the weight average molecular weight of PHA to decrease it. The upper temperature limit at which the biomass is heated is 500° C. The lower limit of the heating temperature is 40° C., preferably 50° C., more preferably 60° C., particularly preferably 70° C., extremely preferably 80° C., most preferably 90° C. If the heating temperature is lower than 40° C., the weight average molecular weight of PHA is not sufficiently decreased, thereby causing problems in productivity and costs. On the other hand, if the heating temperature exceeds 500° C., it becomes impossible to control the weight average molecular weight of PHA.

The upper limit of heating time is preferably 240 hours. The lower limit of the heating time is preferably 1 minute, more preferably 10 minutes, even more preferably 20 minutes, particularly preferably 30 minutes, extremely preferably 1 hour. Needless to say, in order to control the weight average molecular weight of PHA, the heating time can be controlled according to the heating temperature. If the heating time is less than 1 minute, there is a possibility that the weight average molecular weight of PHA is not sufficiently decreased or that problems in productivity and costs are caused. On the other hand, if the heating time exceeds 240 hours, there is a possibility that it becomes impossible to control the weight average molecular weight of PHA.

Examples of the apparatus used to heat the biomass include, but are not limited to, preferably a spray drier, a constant-temperature vacuum drier, a drum heater, a high-temperature furnace, a ceramic heater, a silicon-rubber heater, a high-frequency continuous heating apparatus, a far-infrared heater, and a microwave heating apparatus. Of course, these apparatuses can be used in combination of two or more of them. It is to be noted that biomass to be used is preferably dried biomass. Biomass can be dried by a well-known method such as the above-described method of heating.

<Treatment (b)>

In the case of treatment (b), in addition to treatment (a), an aprotic organic solvent is added to the biomass, and an obtained mixture is further heated under stirring to thereby control the weight average molecular weight of PHA to decrease it. The upper temperature limit at which the biomass is heated in the aprotic organic solvent is 200° C. The lower limit of the heating temperature is 40° C., preferably 50° C., more preferably 60° C., particularly preferably 70° C., extremely preferably 80° C., most preferably 90° C. If the heating temperature is lower than 40° C., there is a possibility that the weight average molecular weight of PHA is not sufficiently decreased or that problems in productivity and costs are caused. On the other hand, if the heating temperature exceeds 200° C., it becomes impossible to control the weight average molecular weight of PHA.

The upper limit of heating time is preferably 240 hours. The lower limit of the heating time is preferably 1 minute, more preferably 1 hour, even more preferably 2 hours, particularly preferably 3 hours, extremely preferably 4 hours, most preferably 5 hours. Needless to say, in order to control the weight average molecular weight of PHA, the heating time can be controlled according to the heating temperature. If the heating time is less than 1 minute, there is a possibility that the weight average molecular weight of PHA is not sufficiently decreased or that problems in productivity and costs are caused. On the other hand, if the heating time exceeds 240 hours, there is a possibility that it becomes impossible to control the weight average molecular weight of PHA.

It is to be noted that biomass to be used is preferably dried biomass. Biomass can be dried by a well-known method such as the method described with reference to treatment (a).

<Treatment (c)>

In the case of treatment (c), an aprotic organic solvent is added to the biomass, and an obtained mixture is further heated under stirring in the presence of water and/or an alcohol to thereby control the weight average molecular weight of PHA to decrease it. By heating the mixture in the presence of water and/or an alcohol, it is possible to enhance the effect of decreasing the weight average molecular weight of PHA. The upper temperature limit at which the biomass is heated in the aprotic organic solvent in the presence of water and/or an alcohol is 200° C. The lower limit of the heating temperature is 40° C., preferably 50° C., more preferably 60° C., particularly preferably 70° C., extremely preferably 80° C., most preferably 90° C. If the heating temperature is lower than 40° C., the weight average molecular weight of PHA is not sufficiently decreased or problems in productivity and costs are caused. On the other hand, if the heating temperature exceeds 200° C., it becomes impossible to control the weight average molecular weight of PHA.

The upper limit of heating time is preferably 240 hours. The lower limit of the heating time is 1 minute, more preferably 30 minutes, even more preferably 1 hour, particularly preferably 2 hours, extremely preferably 3 hours, most preferably 4 hours.

An amount of water and/or an alcohol to be used in heating is preferably 0.01 to 70 parts by weight, more preferably 0.1 to 50 parts by weight, even more preferably 1 to 30 parts by weight, per 100 parts by weight of the aprotic organic solvent used. Needless to say, the amount of water and/or an alcohol can be controlled according to the heating temperature and the heating time. Conversely, the heating temperature and the heating time can be controlled according to the amount of water and/or an alcohol used. If the amount of water and/or an alcohol in the system is less than 0.01 part by weight or the heating time is less than 1 minute, there is a possibility that the weight average molecular weight of PHA is not sufficiently decreased or that problems in productivity and costs are caused. On the other hand, if the heating time exceeds 240 hours or the amount of water and/or an alcohol exceeds 70 parts by weight, there is a possibility that it becomes impossible to control the weight average molecular weight of PHA.

The alcohol to be used preferably has 1 to 20 carbon atoms, more preferably has 1 to 15 carbon atoms, even more preferably has 1 to 10 carbon atoms. Examples of such an alcohol include methanol, ethanol, propanol, butanol, pentanol, hexanol, heptanol, octanol, nonanol, decanol, and isomers thereof.

It is to be noted that the biomass may be either dried biomass or biomass suspended in water when it is heated before addition of a solvent.

<Treatment (d)>

In the case of treatment (d), the biomass obtained above is not heated before addition of an aprotic organic solvent, but an aprotic organic solvent is added to the biomass, and an obtained mixture is heated under stirring in the same manner as in treatment (b), to thereby control the weight average molecular weight of PHA to decrease it. The upper temperature limit at which the biomass is heated in the aprotic organic solvent is 200° C. The lower limit of the heating temperature is 40° C., preferably 50° C., more preferably 60° C., particularly preferably 70° C., extremely preferably 80° C., most preferably 90° C. If the heating temperature is lower than 40° C., the weight average molecular weight of PHA is not sufficiently decreased or problems in productivity and costs are caused. On the other hand, if the heating temperature exceeds 200° C., it becomes impossible to control the weight average molecular weight of PHA.

The upper limit of heating time is preferably 240 hours. The lower limit of the heating time is preferably 1 minute, more preferably 1 hour, even more preferably 2 hours, particularly preferably 3 hours, extremely preferably 4 hours, most preferably 5 hours. Needless to say, in order to control the molecular weight of PHA, the heating time can be controlled according to the heating temperature. If the heating time is less than 1 minute, there is a possibility that the weight average molecular weight of PHA is not sufficiently decreased or that problems in productivity and costs are caused. On the other hand, if the heating time exceeds 240 hours, there is a possibility that it becomes impossible to control the weight average molecular weight of PHA.

An apparatus used to heat the biomass may be the same as that described with reference to treatment (a). It is to be noted that biomass to be used is dried biomass. Biomass can be dried by a well-known method such as the method described with reference to treatment (a).

<Treatment (e)>

In the case of treatment (e), an aprotic organic solvent is added to biomass in the presence of water and/or an alcohol, and then an obtained mixture is heated under stirring in the presence of water and/or an alcohol to thereby control the weight average molecular weight of PHA to decrease it. By heating the mixture in the presence of water and/or an alcohol, it is possible to enhance the effect of decreasing the weight average molecular weight of PHA. The upper temperature limit at which the biomass is heated in the aprotic organic solvent in the presence of water and/or an alcohol is 200° C. The lower limit of the heating temperature is 40° C., preferably 50° C., more preferably 60° C., particularly preferably 70° C., extremely preferably 80° C., most preferably 90° C. If the heating temperature is lower than 40° C., the weight average molecular weight of PHA is not sufficiently decreased or problems in productivity and costs are caused. On the other hand, if the heating temperature exceeds 200° C., it becomes impossible to control the weight average molecular weight of PHA.

The upper limit of heating time is preferably 240 hours. The lower limit of the heating time is 1 minute, more preferably 30 minutes, even more preferably 1 hour, particularly preferably 2 hours, extremely preferably 3 hours, most preferably 4 hours.

An amount of water and/or an alcohol to be used in heating is preferably 0.01 to 70 parts by weight, more preferably 0.1 to 50 parts by weight, even more preferably 1 to 30 parts by weight, per 100 parts by weight of the aprotic organic solvent used. Needless to say, the amount of water and/or an alcohol can be controlled according to the heating temperature and the heating time. Conversely, the heating temperature and the heating time can be controlled according to the amount of water and/or an alcohol used. If the amount of water and/or an alcohol existing in the system is less than 0.01 part by weight or the heating time is less than 1 minute, there is a possibility that the weight average molecular weight of PHA is not sufficiently decreased or that problems in productivity and costs are caused. On the other hand, if the heating time exceeds 240 hours, there is a possibility that it becomes impossible to control the weight average molecular weight of PHA.

The alcohol to be used preferably has 1 to 20 carbon atoms, more preferably has 1 to 15 carbon atoms, even more preferably has 1 to 10 carbon atoms. Examples of such an alcohol include methanol, ethanol, propanol, butanol, pentanol, hexanol, heptanol, octanol, nonanol, decanol, and isomers thereof.

It is to be noted that biomass to be used may be either biomass suspended in water or dried biomass. Biomass can be dried by a well-known method such as the method described with reference to treatment (a).

By carrying out any one of the treatments (a) to (e) described above, the weight average molecular weight of PHA is decreased. Specifically, the weight average molecular weight of PHA is decreased to 2,000,000 or less, preferably 1,750,000 or less, more preferably 1,500,000 or less, but is preferably 500,000 or more. As described above, in a case where the weight average molecular weight of PHA exceeds 2,000,000, there is a problem that the viscosity of a liquid becomes extremely high during extraction so that it is impossible to stir the liquid or the filterability becomes very poor when an extraction residue is separated by filtration. However, the present invention makes it possible to decrease the molecular weight of PHA, and is therefore significantly effective at solving such a problem.

A liquid obtained by carrying out any one of the above-described treatments (a) to (e) is transferred to a filter kept at a predetermined temperature, such as a jacket-type pressure filter, and is then filtered to recover a PHA solution. Specifically, an extraction residue is separated by filtration from a liquid containing extracted PHA whose weight average molecular weight has been decreased through any one of the above-described treatments (a) to (e), and then a poor solvent is added to a PHA solution to crystallize PHA. Preferred examples of a poor solvent include, but are not limited to, aliphatic hydrocarbons having 6 to 12 carbon atoms such as hexane, heptane, methylcyclohexane, octane, nonane, decane, dodecane, undecane, and isomers thereof. At least one of them can be used as a poor solvent. In a case where a solvent having a high affinity for water, such as acetone, is used as an extracting solvent, it is possible to add water as a poor solvent to crystallize PHA.

Crystallized PHA can be recovered by a method well known to those skilled in the art, that is, by separating a liquid from a slurry containing crystallized PHA by means of filtration or centrifugation. The recovered PHA can be washed with a solvent selected from among the extracting solvent and the poor solvent. However, a solvent for washing PHA is not limited thereto, and the recovered PHA can also be washed with a solvent such as water, methanol, ethanol, acetone, hexane, or a mixture of two or more of them. The PHA can be dried by a method well known to those skilled in the art, such as flash drying or vacuum drying.

In the present invention, hydroxyalkanoates constituting PHA are not particularly limited, and specific examples of the hydroxyalkanoate include 3-hydroxybutyrate (3HB), 3-hydroxyvalerate (3HV), 3-hydroxypropionate, 4-hydroxybutyrate, 4-hydroxyvalerate, 5-hydroxyvalerate, 3-hydroxyhexanoate (3HH), 3-hydroxyheptanoate, 3-hydroxyoctanoate, 3-hydroxynonanoate, 3-hydroxydecanoate, and the like. In the present invention, PHA may be either a homopolymer of such a hydroxyalkanoate or a copolymer obtained by copolymerization of two or more such hydroxyalkanoates, but is preferably a copolymer. Specific examples of PHA include PHB that is a homopolymer of 3HB, PHBV that is a copolymer of two monomers, 3HB and 3HV, PHBH (poly(3-hydroxybutyrate-co-3-hydroxyhexanoate)) that is a copolymer of two monomers, 3HB and 3HH (see Japanese Patent No. 2777757), and PHBHV that is a copolymer of three monomers, 3HB, 3HV, and 3HH (see Japanese Patent No. 2777757). Particularly, from the viewpoints of degradability required of a biodegradable polymer and flexibility, copolymers having 3HH as a monomer component are preferable, and PHBH is more preferable. The ratio between the monomer units constituting PHBH is not particularly limited, but from the viewpoint of crystallinity of PHBH, the ratio of 3HH units is preferably 20 mol % or less, more preferably 15 mol % or less, even more preferably 10 mole or less. In the case of PHBHV, the ratio among the monomer units constituting PHBHV is not also particularly limited, but PHBHV of which 3HB unit content is 1 to 95 mol %, 3HV unit content is 1 to 96 mol %, and 3HH unit content is 1 to 30 mol % can be mentioned as a preferred example.

Polyhydroxyalkanoate obtained according to the present invention can be processed into various forms such as various fibers, yarns or threads, ropes, woven fabrics, knitted goods, nonwoven fabrics, papers, films, sheets, tubes, plates or boards, bars or rods, containers, bags, parts, foamed materials, and the like. Further, the polyhydroxyalkanoate can be processed into biaxially-oriented films. These molded products can be used suitably in various fields, for example in agriculture, fishery, forestry, horticulture, medicine, sanitary supplies, clothes, non-clothes, packaging materials, and the like.

According to the present invention, there is also provided a method of producing an extraction residue, which comprises extracting PHA from biomass containing PHA through any one of the above-described treatments (a) to (e) to obtain an extraction residue, and decreasing the amount of the solvent contained in the extraction residue. Although PHA can be extracted from biomass containing PHA by not only the above-described method of producing PHA but also by other well-known methods, the above-described method of producing PHA is employed in the present invention.

A method of decreasing the amount of the solvent contained in the extraction residue is not particularly limited, and examples of such a method include drying by heating, constant-temperature vacuum drying, and drying using a drum heater, a high-temperature furnace, or a far-infrared heater. The extraction residue obtained according to the present invention is preferably used as feed for animals, feed for microorganisms, or fertilizer for plants. Therefore, the solvent content used in the invention is preferably within an acceptable range of solvent content as feed or fertilizer. However, it is more preferred that the solvent is substantially removed from the extraction residue.

The present invention includes feed for animals, feed for microorganisms, and fertilizer for plants which contain the above-described extraction residue.

According to the present invention, it is possible to provide a method of industrially producing PHA cheaply with good productivity, whereby stirring can be carried out without difficulty during extraction of PHA using a solvent from biomass containing PHA having a high weight average molecular weight of such as at least 2,000,000 and filterability of an extraction residue becomes good.

### BEST MODES FOR CARRYING OUT THE INVENTION

Hereinafter, the present invention will be described in more detail with reference to the following Examples. In all Examples, a copolyester, PHBH obtained by polymerization was used. The technical scope of the present invention is not limited to these Examples, and is not limited to PHBH.

It is to be noted that in the following Examples, the weight average molecular weight of PHBH was measured using a gel chromatography system with RI detection (manufactured by Shimadzu Corporation) equipped with two columns of Shodex K806L (manufactured by Showa Denko Co., Ltd., 300×8 mm) connected in series. Chloroform was used as a mobile phase. As a molecular weight standard sample, commercially available standard polystyrene was used. The purity of PHBH was measured by methyl-esterifying PHBH and then subjecting the methyl-esterified PHBH to gas chromatography. A water content was measured using an infrared moisture meter FD-230 manufactured by Kett Electric Laboratory.

### EXAMPLE 1

### Recovery of PHA Through Treatment (a)

Dried biomass (Ralstonia eutropha; weight average molecular weight: 3,000,000; PHBH content: 60% by weight; 3-hydroxyhexanoate (hereinafter abbreviated as “3HH”) unit: 3 mol %; water content: 0.8%) was heated in an oven at 130° C. for 1 hour. Then, 24.8 g of the biomass and 700 g of chloroform (which is an aprotic organic solvent) were placed in a flask, and they were heated at 30° C. for 2 hours. At this time, the biomass and chloroform were very smoothly stirred. The thus obtained mixture was transferred to a jacket-type pressure filter kept at 30° C., and was then filtered to recover a PHBH solution. At this time, the filterability of the mixture was very good. The recovered PHBH solution was kept at 30° C., and 1,400 g of hexane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and hexane, and was then vacuum-dried at 45° C. The amount of the recovered PHBH was 14.1 g (95%), the purity was 99% or more, and the 3HH unit content was 3 mol %. After the completion of such treatment described above, the weight average molecular weight of PHBH was decreased to 1,400,000. The results are shown in Table 1.

### EXAMPLE 2

### Recovery of PHA Through Treatment (b)

Dried biomass used in Example 1 was heated in an oven at 130° C. for 1 hour. Then, 24.8 g of the biomass and 211.4 g of toluene were placed in a flask, and they were heated at 100° C. for 1 hour. At this time, the biomass and toluene were very smoothly stirred. The thus obtained mixture was transferred to a jacket-type pressure filter kept at 100° C., and was then filtered to recover a PHBH solution. At this time, filterability of the mixture was very good. The recovered PHBH solution was kept at 90° C., and 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.1 g (95%), the purity was 99% or more, and the 3HH unit content was 3 mol %. After the completion of such treatment described above, the weight average molecular weight of PHBH was decreased to 1,300,000. The results are shown in Table 1.

### COMPARATIVE EXAMPLE 1

Dried biomass used in Example 1 was not heated, and 24.8 g of the biomass and 700 g of chloroform (which is an aprotic organic solvent) were placed in a flask to carry out extraction at 30° C. for 2 hours. At this time, fluidity of the mixture was low, and therefore it was very difficult to stir the mixture. Then, the mixture was transferred to a jacket-type pressure filter kept at 30° C. for the purpose of recovering a PHBH solution by filtration. However, filterability of the mixture was very poor and therefore it was impossible to recover a PHBH solution. The weight average molecular weight of PHBH was still 3,000,000, that is, the weight average molecular weight was not decreased at all. The results are shown in Table 1.

### COMPARATIVE EXAMPLE 2

Dried biomass used in Example 1 was heated in an oven at 30° C. for 10 hours. Then, 24.8 g of the biomass and 700 g of chloroform (which is an aprotic organic solvent) were placed in a flask to carry out extraction at 30° C. for 2 hours. At this time, fluidity of the mixture was low, and therefore it was very difficult to stir the mixture. Then, the mixture was transferred to a jacket-type pressure filter kept at 30° C. for the purpose of recovering a PHBH solution by filtration. However, filterability of the mixture was very poor, and therefore it was impossible to recover a PHBH solution. The weight average molecular weight of PHBH was still 3,000,000, that is, the weight average molecular weight was not decreased at all. The results are shown in Table 1.

### EXAMPLE 3

### Recovery of PHA Through Treatment (b)

Dried biomass used in Example 1 was heated in an oven at 130° C. for 1 hour. Then, 24.8 g of the biomass and 211.4 g of toluene were placed in a flask, and they were heated at 100° C. for 10 hours. At this time, fluidity of the mixture was high. The mixture was transferred to a jacket-type pressure filter kept at 100° C., and was then filtered to recover a PHBH solution. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 3 mol %. After the completion of such treatment described above, the weight average molecular weight of PHBH was decreased to 900,000. The results are shown in Table 1.

### COMPARATIVE EXAMPLE 3

Dried biomass used in Example 1 was heated in an oven at 30° C. for 10 hours. Then, 24.8 g of the biomass and 700 g of chloroform (which is an aprotic organic solvent) were placed in a flask, and then they were heated at 30° C. for 10 hours. At this time, fluidity of the mixture was low, and therefore it was very difficult to stir the mixture. Then, the mixture was transferred to a jacket-type pressure filter kept at 30° C. for the purpose of recovering a PHBH solution by filtration. However, filterability of the mixture was very poor, and therefore it was impossible to recover a PHBH solution. The weight average molecular weight of PHBH was still 3,000,000, that is, the weight average molecular weight was not decreased at all. The results are shown in Table 1.

### EXAMPLE 4

### Recovery of PHA Through Treatment (c)

Dried biomass used in Example 1 was heated in an oven at 130° C. for 1 hour. Then, 24.8 g of the biomass and 211.4 g of toluene were placed in a flask, 2.0 g of water was added thereto, and then they were heated at 100° C. for 10 hours. At this time, fluidity of the mixture was high. The mixture was transferred to a jacket-type pressure filter kept at 100° C., and was then filtered to recover a PHBH solution. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.1 g (95%), the purity was 99% or more, and the 3HH unit content was 3 mol %. After the completion of such treatment described above, the weight average molecular weight of PHBH decreased to 500,000. The results are shown in Table 1.

### COMPARATIVE EXAMPLE 4

Dried biomass used in Example 1 was heated in an oven at 30° C. for 10 hours. Then, 24.8 g of the biomass and 700 g of chloroform (which is an aprotic organic solvent) were placed in a flask, 0.01 g of water was added thereto, and then they were heated at 30° C. for 10 hours. At this time, fluidity of the mixture was low, and therefore it was very difficult to stir the mixture. Then, the mixture was transferred to a jacket-type pressure filter kept at 30° C. for the purpose of recovering a PHBH solution by filtration. However, filterability of the mixture was very poor, and therefore it was impossible to recover a PHBH solution. The weight average molecular weight of PHBH was still 3,000,000, that is, the weight average molecular weight was not decreased at all. The results are shown in Table 1.

### EXAMPLE 5

### Recovery of PHA Through Treatment (c)

Dried biomass used in Example 1 was heated in an oven at 130° C. for 1 hour. Then, 24.8 g of the biomass and 211.4 g of toluene were placed in a flask, 2.0 g of methanol was added thereto, and then they were heated at 100° C. for 10 hours. At this time, fluidity of the mixture was high. The mixture was transferred to a jacket-type pressure filter kept at 100° C., and was then filtered to recover a PHBH solution. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 3 mol %. After the completion of such treatment described above, the weight average molecular weight of PHBH was decreased to 500,000. The results are shown in Table 1.

### COMPARATIVE EXAMPLE 5

Dried biomass used in Example 1 was heated in an oven at 30° C. for 10 hours. Then, 24.8 g of the biomass and 700 g of chloroform (which is an aprotic organic solvent) were placed in a flask, 0.01 g of methanol was added thereto, and then they were heated at 30° C. for 10 hours. Fluidity of the mixture was low, and therefore it was very difficult to stir the mixture. Then, the mixture was transferred to a jacket-type pressure filter kept at 30° C. for the purpose of recovering a PHBH solution by filtration. However, filterability of the mixture was very poor, and therefore it was impossible to recover a PHBH solution. The weight average molecular weight of PHBH was still 3,000,000, that is, the weight average molecular weight was not decreased at all. The results are shown in Table 1.

### EXAMPLE 6

### Recovery of PHA Through Treatment (d)

Dried biomass used in Example 1 was not heated, 24.8 g of the biomass and 211.4 g of toluene were placed in a flask, and then they were heated at 100° C. for 10 hours. At this time, fluidity of the mixture was high. Then, the mixture was transferred to a jacket-type pressure filter kept at 100° C. to recover a PHBH solution by filtration. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.1 g (95%), the purity was 99% or more, and the 3HH unit content was 3 mol %. After the completion of such treatment described above, the weight average molecular weight of PHBH was decreased to 1,500,000. The results are is shown in Table 1.

### EXAMPLE 7

### Recovery of PHA Through Treatment (e)

Dried biomass used in Example 1 was not heated, 24.8 g of the biomass and 211.4 g of toluene were placed in a flask, 2.0 g of water was added thereto, and then they were heated at 100° C. for 10 hours. At this time, fluidity of the mixture was high. Then, the mixture was transferred to a jacket-type pressure filter kept at 100° C. to recover a PHBH solution by filtration. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 3 mol %. After the completion of such treatment described above, the weight average molecular weight of PHBH was decreased to 1,000,000. The results are shown in Table 1.

### EXAMPLE 8

### Recovery of PHA Through Treatment (e)

Dried biomass used in Example 1 was not heated, 24.8 g of the biomass and 211.4 g of toluene were placed in a flask, 2.0 g of methanol was added thereto, and then they were heated at 100° C. for 10 hours. At this time, fluidity of the mixture was high. Then, the mixture was transferred to a jacket-type pressure filter kept at 100° C. to recover a PHBH solution by filtration. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 3 mol %. After the completion of such treatment described above, the weight average molecular weight of PHBH was decreased to 1,000,000. The results are shown in Table 1.

### EXAMPLE 9

### Recovery of PHA Through Treatment (a)

Dried biomass (Ralstonia eutropha; weight average molecular weight: 2,200,000; PHBH content: 60% by weight; 3-hydroxyhexanoate (hereinafter abbreviated as “3HH”) unit: 7 mol %; water content: 0.9%) was heated in an oven at 50° C. for 120 hours. Then, 24.8 g of the biomass and 700 g of chloroform (which is an aprotic organic solvent) were placed in a flask, and they were heated at 30° C. for 2 hours. At this time, the biomass and chloroform were smoothly stirred. The thus obtained mixture was transferred to a jacket-type pressure filter kept at 30° C., and was then filtered to recover a PHBH solution. At this time, filterability of the mixture was good. The recovered PHBH solution was kept at 30° C., and 1,400 g of hexane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and hexane, and was then vacuum-dried at 45° C. The amount of the recovered PHBH was 14.0 g (94%), the purity of the PHBH was 99% or more, and the 3HH unit content was 7 mol %. After the completion of such treatment described above, the weight average molecular weight of PHBH was decreased to 1,800,000. The results are shown in Table 1.

### EXAMPLE 10

### Recovery of PHA Through Treatment (b)

Dried biomass used in Example 9 was heated in an oven at 50° C. for 120 hours. Then, 24.8 g of the biomass and 211.4 g of toluene were placed in a flask, and they were heated at 50° C. for 120 hours. At this time, the biomass and toluene were very smoothly stirred. The thus obtained mixture was transferred to a jacket-type pressure filter kept at 100° C., and was then filtered to recover a PHBH solution. At this time, filterability of the mixture was very good. The recovered PHBH solution was kept at 90° C., and 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.1 g (95%), the purity was 99% or more, and the 3HH unit content was 7 mol %. After the completion of such treatment described above, the weight average molecular weight of PHBH was decreased to 1,200,000. The results are shown in Table 1.

### EXAMPLE 11

### Recovery of PHA Through Treatment (c)

Dried biomass used in Example 9 was heated in an oven at 50° C. for 120 hours. Then, 24.8 g of the biomass and 211.4 g of toluene were placed in a flask, 2.0 g of water was added thereto, and they were heated at 50° C. for 120 hours. At this time, fluidity of the mixture was high. The thus obtained mixture was transferred to a jacket-type pressure filter kept at 100° C., and was then filtered to recover a PHBH solution. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 7 mol %. After the completion of such treatment described above, the weight average molecular weight of PHBH was decreased to 700,000. The results are shown in Table 1.

### EXAMPLE 12

### Recovery of PHA Through Treatment (d)

Dried biomass used in Example 9 was not heated, 24.8 g of the biomass and 211.4 g of toluene were placed in a flask, and then they were heated at 50° C. for 120 hours. At this time, fluidity of the mixture was high. The thus obtained mixture was subjected to extraction at 100° C. for 1 hour, and was then transferred to a jacket-type pressure filter kept at 100° C. to recover a PHBH solution by filtration. At this time, filterability of the mixture was very good. The recovered PHBH solution was kept at 90° C., and then 210 gof heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.1 g (95%), the purity was 99% or more, and the 3HH unit content was 7 mol %. After the completion of such treatment described above, the weight average molecular weight of PHBH was decreased to 1,600,000. The results are shown in Table 1.

### EXAMPLE 13

### Recovery of PHA Through Treatment (e)

Dried biomass used in Example 9 was not heated, 24.8 g of the biomass and 211.4 g of toluene were placed in a flask, 2.0 g of water was added thereto, and then they were heated at 50° C. for 120 hours. At this time, fluidity of the mixture was high. The thus obtained mixture was subjected to extraction at 100° C. for 1 hour, and was then transferred to a jacket-type pressure filter kept at 100° C. to recover a PHBH solution by filtration. At this time, filterability of the mixture was very good. The recovered PHBH solution was kept at 90° C., and then 210 g of heptane was added little by little thereto while the solution was strongly stirred. As a result, white PHBH was precipitated. Then, the solution was cooled to room temperature. The precipitated PHBH was easily recovered by filtration. The recovered PHBH was washed with 50 g of a mixed solvent of equal parts of toluene and heptane, and was then vacuum-dried at 45° C. The amount of the recovered PHBH was 14.0 g (94%), the purity was 99% or more, and the 3HH unit content was 7 mol %. After the completion of such treatment described above, the weight average molecular weight of PHBH was decreased to 1,100,000. The results are shown in Table 1.

TABLE 1

| | | Examples | | | | | | | | |
|---|---|---|---|---|---|---|---|---|---|---|
| | | 1 | 2 | 3 | 4 | 5 | 6 | 7 | 8 | 9 |
| Treatment method | | (a) | (b) | (b) | (c) | (c) | (d) | (e) | (e) | (a) |
| Before addition of | Treatment temperature (° C.) | 130 | 130 | 130 | 130 | 130 | — | — | — | 50 |
| aprotic organic solvent | Treatment time (Hr) | 1 | 1 | 1 | 1 | 1 | — | — | — | 120 |
| After addition of | Treatment temperature (° C.) | 30 | 100 | 100 | 100 | 100 | 100 | 100 | 100 | 30 |
| aprotic organic solvent | Treatment time (Hr) | 2 | 1 | 10 | 10 | 10 | 10 | 10 | 10 | 2 |

| | | | | | | | | | |
|---|---|---|---|---|---|---|---|---|---|
| Water or alcohol | — | — | — | Water | Methanol | — | Water | Methanol | — |
| Molecular weight before treatment | 300 | 300 | 300 | 300 | 300 | 300 | 300 | 300 | 220 |
| (Unit: ten thousands) | | | | | | | | | |
| Molecular weight after treatment | 140 | 130 | 90 | 50 | 50 | 150 | 100 | 100 | 180 |
| (Unit: ten thousands) | | | | | | | | | |

| | | Examples | | | | | Comparative Examples | | | | |
|---|---|---|---|---|---|---|---|---|---|---|---|
| | | 10 | 11 | 12 | | 13 | 1 | 2 | 3 | 4 | 5 |
| Treatment method | | (b) | (c) | (d) | | (e) | — | — | — | — | — |
| Before addition of | Treatment temperature (° C.) | 50 | 50 | — | | — | — | 30 | 30 | 30 | 30 |
| aprotic organic solvent | Treatment time (Hr) | 120 | 120 | — | | — | — | 10 | 10 | 10 | 10 |
| After addition of | Treatment temperature (° C.) | 50 | 50 | 50 | 100 | 50 | 30 | 30 | 30 | 30 | 30 |
| aprotic organic solvent | Treatment time (Hr) | 120 | 120 | 120 | 1 | 120 | 2 | 2 | 10 | 10 | 10 |

| | | | | | | | | | |
|---|---|---|---|---|---|---|---|---|---|
| Water or alcohol | — | Water | — | Water | — | — | — | Water | Methanol |
| Molecular weight before treatment | 220 | 220 | 220 | 220 | 300 | 300 | 300 | 300 | 300 |
| (Unit: ten thousands) | | | | | | | | | |
| Molecular weight after treatment | 120 | 70 | 160 | 110 | 300 | 300 | 300 | 300 | 300 |
| (Unit: ten thousands) | | | | | | | | | |

1. A method of producing polyhydroxyalkanoate by extracting and isolating polyhydroxyalkanoate by using an aprotic organic solvent from biomass containing polyhydroxyalkanoate having a weight average molecular weight of more than 2,000,000,
which comprises decreasing the weight average molecular weight of the polyhydroxyalkanoate through any one of the following treatments:
(a) the biomass is heated at 40 to 500° C. before addition of the aprotic organic solvent;
(b) the biomass is heated at 40 to 500° C. before addition of the aprotic organic solvent, and is further heated at 40 to 200° C. in the aprotic organic solvent;
(c) the biomass is heated at 40 to 500° C. before addition of the aprotic organic solvent, and is further heated at 40 to 200° C. in the aprotic organic solvent in the presence of water and/or an alcohol;
(d) the biomass is not heated before addition of the aprotic organic solvent, but is heated at 40 to 200° C. in the aprotic organic solvent; and
(e) the biomass is not heated before addition of the aprotic organic solvent, but is heated at 40 to 200° C. in the aprotic organic solvent in the presence of water and/or an alcohol.

2. The method of producing polyhydroxyalkanoate according to claim 1,
wherein the aprotic organic solvent is at least one species selected from the group consisting of aromatic hydrocarbons having 6 to 10 carbon atoms, ketones having 3 to 7 carbon atoms, and fatty acid alkyl esters having 4 to 8 carbon atoms.

3. The method of producing polyhydroxyalkanoate according to claim 2,
wherein the aromatic hydrocarbons having 6 to 10 carbon atoms is at least one species selected from the group consisting of benzene, chlorobenzene, toluene, xylene, ethylbenzene, cumene, butylbenzene, cymene, and isomers thereof.

4. The method of producing polyhydroxyalkanoate according to claim 2,
wherein the ketone having 3 to 7 carbon atoms is at least one species selected from the group consisting of acetone, methylethylketone, methylbutylketone, pentanone, hexanone, cyclohexanone, heptanone, and isomers thereof.

5. The method of producing polyhydroxyalkanoate according to claim 2,
wherein the fatty acid alkyl ester having 4 to 8 carbon atoms is at least one species selected from the group consisting of ethylacetate, propylacetate, butylacetate, pentylacetate, hexylacetate, and isomers thereof.

6. The method of producing polyhydroxyalkanoate according to claim 1,
wherein the biomass is heated for 1 minute to 240 hours before addition of the aprotic organic solvent.

7. The method of producing polyhydroxyalkanoate according to claim 1,
Wherein the biomass is heated in the aprotic organic solvent for 1 minute to 240 hours.

8. The method of producing polyhydroxyalkanoate according to claim 1,
wherein the biomass is heated in the aprotic organic solvent in the presence of water and/or an alcohol for 1 minute to 240 hours.

9. The method of producing polyhydroxyalkanoate according to claim 8,
wherein the amount of water and/or an alcohol is 0.01 to 70 parts by weight per 100 parts by weight of the aprotic organic solvent.

10. The method of producing polyhydroxyalkanoate according to claim 9,
wherein the alcohol has 1 to 20 carbon atoms.

11. The method of producing polyhydroxyalkanoate according to claim 10,
wherein the alcohol is at least one species selected from the group consisting of methanol, ethanol, propanol, butanol, pentanol, hexanol, heptanol, octanol, nonanol, decanol, and isomers thereof.

12. The method of producing polyhydroxyalkanoate according to claim 1,
wherein the polyhydroxyalkanoate is a copolymer obtained by copolymerizing at least two monomers selected from the group consisting of 3-hydroxybutylate, 3-hydroxyvalerate, 3-hydroxypropionate, 4-hydroxybutylate, 4-hydroxyvalerate, 5-hydroxyvalerate, 3-hydroxyhexanoate, 3-hydroxyheptanoate, 3-hydroxyoctanoate, 3-hydroxynonanoate, and 3-hydroxydecanoate.

13. The method of producing polyhydroxyalkanoate according to claim 12,
wherein the polyhydroxyalkanoate is a copolymer comprising 3-hydroxyhexanoate and at least one hydroxyalkanoate other than 3-hydroxyhexanoate.

14. The method of producing polyhydroxyalkanoate according to claim 13,
wherein the polyhydroxyalkanoate is a copolymer comprising 3-hydroxyhexanoate and 3-hydroxybutylate.

15. The method of producing polyhydroxyalkanoate according to claim 1,
wherein the polyhydroxyalkanaote is produced by a microorganism belonging to a genus selected from the group consisting of Alcaligenes, Azotobacter, Bacillus, Clostridium, Halobacterium, Norcadia, Rhodospirillum, Pseudomonas, Ralstonia, Zoogloea, Candida, Yarrowia, Saccharomyces, and Aeromonas.

16. The method of producing polyhydroxyalkanoate according to claim 1,
wherein the polyhydroxyalkanoate is produced by a transformant into which a polyhydroxyalkanoate synthase gene cluster derived from Aeromonas caviae has been introduced.

17. The method of producing polyhydroxyalkanoate according to claim 16,
wherein the polyhydroxyalkanoate is produced by Ralstonia eutropha into which a polyhydroxyalkanoate synthase gene cluster derived from Aeromonas caviae has been introduced.

18. A method of producing an extraction residue by extracting and isolating polyhydroxyalkanoate by using an aprotic organic solvent from biomass containing polyhydroxyalkanoate having a weight average molecular weight of more than 2,000,000,
which comprises extracting the polyhydroxyalkanaote from the biomass through any one of the following treatments to obtain an extraction residue:
(a) the biomass is heated at 40 to 500° C. before addition of the aprotic organic solvent;
(b) the biomass is heated at 40 to 500° C. before addition of the aprotic organic solvent, and is further heated at 40 to 200° C. in the aprotic organic solvent;
(c) the biomass is heated at 40 to 500° C. before addition of the aprotic organic solvent, and is further heated at 40 to 200° C. in the aprotic organic solvent in the presence of water and/or an alcohol;
(d) the biomass is not heated before addition of the aprotic organic solvent, but is heated at 40 to 200° C. in the aprotic organic solvent; and
(e) the biomass is not heated before addition of the aprotic organic solvent, but is heated at 40 to 200° C. in the aprotic organic solvent in the presence of water and/or an alcohol: and
decreasing the amount of the solvent contained in the extraction residue.

19. Feed for animals, feed for microorganisms, and fertilizer for plants,
which contain an extraction residue produced by the method according to claim 18.
